(19) United States
(12) Patent Application Publication
(10) Pub. No.: US 2025/0041320 A1
(43) Pub. Date: Feb. 6, 2025

(54) USE OF OLIGOSACCHARIDE PREPARATION TO INCREASE PERFORMANCE OF ANIMAL VACCINATED AGAINST COCCIDIOSIS

(71) Applicant: DSM IP ASSETS B.V., Heerlen (NL)

(72) Inventors: Elkin AMAYA, Getzersdorf (AT); Britt BLOKKER, Getzersdorf (AT); Cristiano BORTOLUZZI, Getzersdorf (AT); John GERRMIA, Getzersdorf (AT); Alex KARAGIANNIS-VOULES, Getzersdorf (AT); Estefania PEREZ CALVO, Getzersdorf (AT); Jose-Otavio SORBARA, Getzersdorf (AT)

(21) Appl. No.: 18/718,648

(22) PCT Filed: Dec. 9, 2022
(86) PCT No.: PCT/EP2022/085115

§ 371 Date: Jun. 11, 2024

Related U.S. Application Data
(60) Provisional application No. 63/337,712, filed on May 3, 2022.

(30) Foreign Application Priority Data
Dec. 13, 2021 (EP) 21214207.9

(51) Int. Cl. A61K 31/702; A23K 20/163; A23K 50/75; A61P 3/02

(52) U.S. Cl. CPC A61K 31/702; A23K 20/163; A23K 50/75; A61P 3/02

(57) ABSTRACT

The present invention relates to use of oligosaccharide preparations to increase average body weight and/or to decrease feed conversion ratio and/or to increase average body weight gain of a bird, and to oligosaccharide preparations for use in treatment, amelioration and/or prophylaxis of a bird vaccinated against coccidiosis.

The present invention relates to use of oligosaccharide preparations to increase average body weight and/or to decrease feed conversion ratio and/or to increase average body weight gain of a bird, and to oligosaccharide preparations for use in treatment, amelioration and/or prophylaxis of a bird vaccinated against coccidiosis.

Effects achievable by administration of oligosaccharide preparations to animals are known e.g. from WO 2020/097458 A1.

Coccidia are obligate intracellular parasitic protozoa described to infect mammals, birds, fish, reptiles as well as amphibians. Most coccidian species are host-specific. Notwithstanding, Toxoplasma gondii is known to infect a large range of hosts, in particular mammals, causing toxoplasmosis.

Upon infection of a host's intestinal tract by coccidia, symptoms such as internal hemorrhage, diarrhea, necroses, inflammations etc. may occur. As a consequence thereof, animals suffering from coccidiosis can be found to show loss of appetite, emaciation, fatigue, lethargy, general weakness. Ultimately and particularly in the case of young animals, coccidiosis is known to cause considerably increased mortality. For instance, caecal coccidiosis may result in a mortality of 80% in young chicks.

In livestock farming, in particular in poultry production, coccidian infection leads not only to animal suffering but also to economic losses for the agriculturist. Poultry coccidiosis is caused by coccidia of the genus Eimeria, in particular by E. tenella and E. necatrix, but also e.g. E. acervulina, E. brunetti, E. hagani, E. maxima, E. mivati, E. mitis, E. praecox, E. adenoeides, E. dispersa, E. gallopavonis, E. innocus, E. meleagridis, E. meleagrmitis, E. subrotunda, E. grenier, E. colchici.

Nowadays' poultry raising only takes a few weeks. Commonly, the birds are slaughtered at 6 weeks from hatch. From hatch to slaughter, current chicken breeds are raised to show a fast increase in body weight. In particular in weeks 2 to 4, maximum body weight increase is expected. Due to the parasitic cycle of the relevant Eimeria species of 6 to 8 days, birds infected by these coccidia are affected the most in said weeks of maximum body weight increase, resulting in severe growth depression and thus economic losses.

In addition, in case a bird survives a first infection by a first Eimeria species, the large number of further Eimeria species capable of infecting poultry leads to a situation wherein another infectious cycle might start immediately after the first infection. As a consequence, the affected birds suffer from chronic coccidiosis. Owing to present-day intensive farming methods, coccidia infection spreads fast through an entire flock and may persist on site even for years.

Prophylactic measures to avoid coccidia are limited to rigorous hygiene and desinfection measures. Notwithstanding and despite good husbandry practice, the presence of coccidian parasites can barely be avoided entirely. In addition to said prophylactic measures, it is thus common practice to treat the animals with coccidiostatic chemicals and/or antibiotics such as sulfonamides or triazines. However, due to regulatory requirements and ever increasing public demand, treatment with such coccidiostats may be substituted by vaccination against coccidiosis, e.g. a vaccine from the commercially available COCCIVAC® series.

Upon vaccination against coccidiosis however, birds are prone to show a decrease in performance in comparison to non-vaccinated birds, in particular in the first week(s) after vaccination. In particular, birds vaccinated against coccidiosis can be found to show decreased average body weight, increased feed conversion ratio and/or decreased average body weight gain, in particular in the first week(s) after vaccination, when compared to birds not vaccinated against coccidiosis.

In view of the prior art as outlined above, it is an objective of the present invention to counteract adverse effect(s) of vaccination against coccidiosis. In other words, it is an objective of the present invention to bring the performance of birds vaccinated against coccidiosis to the performance which would be achieved if the same birds were not vaccinated against coccidiosis. It is also an objective to improve the performance of birds vaccinated against coccidiosis.

Surprisingly, this objective has been found to be achieved by a use of an oligosaccharide preparation (e.g. a synthetic oligosaccharide preparation) to increase average body weight and/or to decrease feed conversion ratio and/or to increase average body weight gain of a bird vaccinated against coccidiosis, wherein the oligosaccharide preparation comprises at least n fractions of oligosaccharides each having a distinct degree of polymerization selected from 1 to n (DP1 to DPn fractions), wherein n is an integer greater than or equal to 2; wherein each fraction comprises from at least about 0.5% to about 90% (e.g. from 1% to 90%; or e.g. from about 0.5% to about 15%) of anhydro-subunit containing oligosaccharides by relative abundance as determined by mass spectrometry; and wherein the average body weight of the bird is increased and/or the feed conversion ratio of the bird is decreased and/or the average body weight gain of the bird is increased, all in comparison to a comparable control bird, wherein the comparable control bird was not administered the oligosaccharide preparation. In particular, the invention relates to the use described above, wherein the average body weight of the bird is increased and/or the feed conversion ratio of the bird is decreased and/or the average body weight gain of the bird is increased on at least one day within 42 days after vaccination against coccidiosis (e.g. on one or more day(s) selected from the group of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42 day(s) after vaccination against coccidiosis). Further, the average body weight may be increased and/or the feed conversion ratio may be decreased and/or the average body weight gain may be increased on at least one day 1-42 days after vaccination against coccidiosis (e.g. 25-42 days after vaccination against coccidiosis, and/or 30-42 days after vaccination against coccidiosis). Further in particular, the invention relates to the use described above, wherein the average body weight is increased by at least 0.2% (e.g. at least 0.5%, 0.75%, 1.00%, 1.25%, 1.50%, 1.68%, 1.70%, 2.0%, 2.5%, 2.7% etc.); and/or wherein the feed conversion ratio is decreased by at least 0.2% (e.g. at least 0.5%, 0.75%, 1.00%, 1.25%, 1.50%, 1.7%, 2.0%, 2.4%, 2.5%, 2.7%, 2.8% etc.); and/or wherein the average body weight gain is increased by at least 0.2% (e.g. at least 0.5%, 0.75%, 1.0%, 1.5%, 2%, 2.5%, 2.7%, 2.8%, 3.0%) on at least one day within 42 days after vaccination against coccidiosis (e.g. on one or more day(s) selected from the group of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42 day(s) after vaccination against coccidiosis).

In another aspect, the invention relates to a use of an oligosaccharide preparation (e.g. synthetic oligosaccharide preparation) to adapt the average body weight and/or the feed conversion ratio and/or the average body weight gain of a bird vaccinated against coccidiosis to match the average body weight and/or the feed conversion ratio and/or the average body weight gain of a bird not vaccinated against coccidiosis; wherein the oligosaccharide preparation comprises at least n fractions of oligosaccharides each having a distinct degree of polymerization selected from 1 to n (DP1 to DPn fractions), wherein n is an integer greater than or equal to 2; and wherein each fraction comprises from at least about 0.5% to about 90% (e.g. from 1% to 90%; or e.g. from about 0.5% to about 15%) of anhydro-subunit containing oligosaccharides by relative abundance as determined by mass spectrometry. In other words, the use of the oligosaccharide preparation as described herein allows a bird vaccinated against coccidiosis to show essentially the same performance as if that bird would not have been vaccianted against coccidiosis, thus overcoming the adverse effect(s) of vaccination against coccidiosis. In a particular embodiment of this other aspect, the invention relates to said use, wherein the average body weight and/or the feed conversion ratio and/or the average body weight gain of the bird vaccinated against coccidiosis matches the average body weight and/or the feed conversion ratio and/or the average body weight gain of a bird vaccinated against coccidiosis of the bird not vaccinated against coccidiosis on at least one day within 42 days after vaccination against coccidiosis (e.g. on one or more day(s) selected from the group of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42 day(s) after vaccination against coccidiosis). In a further particular embodiment of this other aspect of the invention, the invention relates to said use, wherein the average body weight of the bird not vaccinated against coccidiosis is at least 0.2% (e.g. at least 0.5%, 0.75%, 1.00%, 1.25%, 1.50%, 1.68%, 1.70%, 2.0%, 2.5%, 2.7% etc.) higher than the average body weight of a bird vaccinated against coccidiosis and not administered the oligosaccharide preparation; and or the feed conversion ratio of the bird not vaccinated against coccidiosis is at least 0.2% (e.g. at least 0.5%, 0.75%, 1.00%, 1.25%, 1.50%, 1.7%, 2.0%, 2.4%, 2.5%, 2.7%, 2.8% etc.) lower than the feed conversion ratio of a bird vaccinated against coccidiosis and not administered the oligosaccharide preparation; and/or the average body weight gain of a bird not vaccinated against coccidiosis is at least 0.2% (e.g. at least 0.5%, 0.75%, 1.0%, 1.5%, 2%, 2.5%, 2.7%, 2.8%, 3.0%) higher than the average body weight gain of a bird vaccinated against coccidiosis and not administered the oligosaccharide preparation on at least one day within 42 days after vaccination against coccidiosis (e.g. on one or more day(s) selected from the group of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42 day(s) after vaccination against coccidiosis).

As a consequence of such use, the performance (i.e. average body weight, feed conversion ratio and/or average body weight gain) of a bird vaccinated against coccidiosis could be improved in comparison to a bird vaccinated against coccidiosis but not administered the oligosaccharide preparation according to a use of the invention. In particular, the performance of a bird vaccinated against coccidiosis and administered the oligosaccharide preparation according to a use of the invention could be improved to reach a level comparable to a bird not vaccinated against coccidiosis.

The oligosaccharide preparation as referred to herein may be administered to the animal before, after, and/or simultaneously, preferably simultaneously, with the diet of the bird vaccinated against coccidiosis.

Merely for clarification, the feed conversion ratio (FCR) is the ratio of mass of feed intake over the mass of animal built as a result of said feed intake. Thus, efficient conversion of food/feed into body mass thus results in a lower FCR, than a less efficient conversion. Consequently, a low FCR is desired. Preferably, the feed conversion ratio as referred to herein is adjusted for mortality. In other words, the FCR is the inverse of the so-called feed efficiency, which feed efficiency is calculated by dividing the body weight or mass by the feed intake weight or mass.

Notably, it was found that while the average body weight, the feed conversion ratio and/or the average body weight gain of birds vaccinated against coccidiosis could be improved when administered the oligosaccharide preparation as compared to birds not administered the oligosaccharide preparation, the mortality rate was found not to be significantly altered. Significance can be statistically evaluated at P<0.05 in a typical ANOVA analysis of variance test model.

In some embodiments of the invention, the oligosaccharide preparation is used as described herein, wherein the oligosaccharide preparation is comprised in a nutritional composition administered to the bird. In a particular embodiment, the oligosaccharide preparation is comprised in the nutritional composition at an inclusion rate of at least 50 ppm (e.g. at least 50, 70, 100, 150, 200, 300, 400, 500 ppm etc.).

In some embodiments of the invention, the bird is poultry (e.g. chicken, duck, goose, turkey, guinea fowl, pigeon, quail), preferably chicken.

In some embodiments of the invention, the oligosaccharide preparation comprises at least n fractions of oligosaccharides each having a distinct degree of polymerization selected from 1 to n (DP1 to DPn fractions) as described above, wherein n is at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100.

In some embodiments, the oligosaccharide preparation as described herein is further characterized in that at least one fraction of the oligosaccharide preparation comprises less than 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, or 2% anhydro-subunit containing oligosaccharides by relative abundance; and/or wherein each fraction of the oligosaccharide preparation comprises greater than 0.2%, 0.5%, 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, or 80% anhydro-subunit containing oligosaccharides by relative abundance.

In some embodiments, the invention relates to a use as described herein, wherein the oligosaccharide preparation has a weight average molecular weight from about 300 to 5000 g/mol (e.g. from about 2000 to 2800 g/mol, 2100 to 2700 g/mol, 2200 to 2600 g/mol, 2300 to 2500 g/mol, or 2320 to 2420 g/mol), 500 to 5000 g/mol, 700 to 5000 g/mol, 500 to 2000 g/mol, 700 to 2000 g/mol, 700 to 1500 g/mol, 300 to 1500 g/mol, 300 to 2000 g/mol, 400 to 1300 g/mol, 400 to 1200 g/mol, 400 to 1100 g/mol, 500 to 1300 g/mol, 500 to 1200 g/mol, 500 to 1100 g/mol, 600 to 1300 g/mol, 600 to 1200 g/mol, or 600 to 1100 g/mol; and/or wherein the oligosaccharide preparation has a number average molecular weight from about 1000 to 2000 g/mol, 1100 to 1900 g/mol, 1200 to 1800 g/mol, 1300 to 1700 g/mol, 1400 to 1600 g/mol, or 1450 to 1550 g/mol.

In some embodiments, the invention relates to a use as described herein, wherein the oligosaccharide preparation is administered to the bird before, after, and/or simultaneously with the diet of the bird vaccinated against coccidiosis, preferably simultaneously with the diet.

In some embodiments, the invention relates to a use as described herein, wherein the relative abundance of oligosaccharides in each of the n fractions of the oligosaccharide preparation decreases monotonically with its degree of polymerization.

It is a further objective of the invention to provide means to support vaccination against coccidiosis in birds. This objective is achieved by providing a oligosaccharide preparation (e.g. synthetic oligosaccharide preparation) for use in treatment, amelioration and/or prophylaxis of a bird vaccinated against coccidiosis, wherein the oligosaccharide preparation comprises at least n fractions of oligosaccharides each having a distinct degree of polymerization selected from 1 to n (DP1 to DPn fractions), wherein n is an integer greater or equal to 2; and wherein each fraction comprises from at least about 0.5% to about 90% (e.g. from 1% to 90%) of anhydro-subunit containing oligosaccharides by relative abundance as determined by mass spectrometry.

In particular, it is an objective to provide means to counteract the adverse effect(s) associated with vaccination against coccidiosis in birds. This objective is achieved by providing an oligosaccharide preparation (e.g. synthetic oligosaccharide preparation) for use in treatment, amelioration and/or prophylaxis of at least one adverse effect of vaccination against coccidiosis in a bird vaccinated against coccidiosis, wherein the oligosaccharide preparation comprises at least n fractions of oligosaccharides each having a distinct degree of polymerization selected from 1 to n (DP1 to DPn fractions), wherein n is an integer greater or equal to 2; and wherein each fraction comprises from at least about 0.5% to about 90% (e.g. from 1% to 90%) of anhydro-subunit containing oligosaccharides by relative abundance as determined by mass spectrometry.

It is a further objective to provide means to support the effect of vaccination against coccidiosis in birds. This further objective is achieved by providing an oligosaccharide preparation (e.g. synthetic oligosaccharide preparation) for use in treatment, amelioration and/or prophylaxis against coccidiosis, wherein the oligosaccharide preparation is used in addition to a vaccination against coccidiosis, wherein the oligosaccharide preparation comprises at least n fractions of oligosaccharides each having a distinct degree of polymerization selected from 1 to n (DP1 to DPn fractions), wherein n is an integer greater or equal to 2; and wherein each fraction comprises from at least about 0.5% to about 90% (e.g. from 1% to 90%; or e.g. from about 0.5% to about 15%) of anhydro-subunit containing oligosaccharides by relative abundance as determined by mass spectrometry. When using such an oligosaccharide preparation for said purpose, an increased efficiency of the vaccination against coccidiosis can be achieved. In particular, a reduction in the median relative abundance of Eimeria species in cecal digesta samples of a bird vaccinated against coccidiosis can be achieved compared to a comparable control bird, which comparable control bird was vaccinated against coccidiosis but not administered the oligosaccharide preparation. Therefore, the invention also relates to a use of an oligosaccharide preparation, e.g. a synthetic oligosaccharide preparation, to reduce the relative abundance of microorganisms from the genus Eimeria in a bird, wherein the oligosaccharide preparation comprises at least n fractions of oligosaccharides each having a distinct degree of polymerization selected from 1 to n (DP1 to DPn fractions), wherein n is an integer greater than or equal to 2; and wherein each fraction comprises from at least about 0.5% to about 90% (e.g. from 1% to 90%; or e.g. from about 0.5% to about 15%) of anhydro-subunit containing oligosaccharides by relative abundance as determined by mass spectrometry. Further, the invention therefore also relates to a use of an oligosaccharide preparation, e.g. a synthetic oligosaccharide preparation, to support, in particular to increase, the anti-coccidiosis effect of a vaccination against coccidiosis (i.e. the reduction of coccidiosis-related and/or -causing microorganisms such as Eimeria), wherein the oligosaccharide preparation comprises at least n fractions of oligosaccharides each having a distinct degree of polymerization selected from 1 to n (DP1 to DPn fractions), wherein n is an integer greater than or equal to 2; and wherein each fraction comprises from at least about 0.5% to about 90% (e.g. from 1% to 90%; or e.g. from about 0.5% to about 15%) of anhydro-subunit containing oligosaccharides by relative abundance as determined by mass spectrometry.

It is also considered that the oligosaccharide preparation is provided in the form of a powderous formulation comprising at least 20% (w/w) of the oligosaccharide preparation as referred to herein; at least 25% (wt/wt) of a silica-based adsorbate (e.g. diatomaceous earth, amorphous precipitated silica) having an average particle size D of less than or equal to 3000 μm (e.g. 100-500, 200-500, 200-300 μm); and optionally 0-25% (wt/wt) of water and/or an auxiliary substance; wherein the % are based on the total weight of the powderous formulation. For instance, such a powderous formulation may comprise 30-70% (wt/wt) of the oligosaccharide preparation as referred to herein; 30-70% (wt/wt) of a silica based adsorbate (e.g. having an average particle size of at least 50 μm); and 0-21% (wt/wt) of water; wherein the % are based on the total weight of the powderous formulation.

Descriptions and methods of manufacturing oligosaccharide preparations according to the invention are described in WO 2020/097458 and WO 2016/007778, in particular in the Examples described therein. In particular, oligosaccharide preparations according to the invention are characterized by the step of heating an aqueous composition comprising one or more feed sugars and a catalyst to a temperature and for a time sufficient to induce polymerization.

Merely for the sake of clarity, the term “oligosaccharide preparation” may refer to a preparation that comprises one or more oligosaccharides.

As used herein, an “oligosaccharide” or “oligomer” may refer to a monosaccharide or a compound containing two or more monosaccharide subunits linked by glycosidic bonds. An “oligosaccharide” may also refer to an anhydro-monosaccharide or a compound containing two or more monosaccharide subunits, where at least one monosaccharide unit is replaced by an anhydro-subunit. An “oligosaccharide” may be optionally functionalized. As used herein, the term “oligosaccharide” encompasses all species of the oligosaccharide, wherein each of the monosaccharide subunit in the oligosaccharide is independently and optionally functionalized and/or replaced with its corresponding anhydro-monosaccharide subunit.

An “anhydro-subunit” may be a product of reversible thermal dehydration of a monosaccharide (or monosaccharide subunit) or a sugar caramelization product. For example, an “anhydro-subunit” may be an anhydro-monosaccharide such as anhydro-glucose. As another example, an “anhydro-subunit” may be linked with one or more regular or anhydro-monosaccharide subunits via glycosidic linkage.

An oligosaccharide may be characterized to contain two or more monosaccharide subunits linked by glycosidic bonds. In this regard, a “gluco-oligosaccharide” may refer to a glucose or a compound containing two or more glucose monosaccharide subunits linked by glycosidic bonds. A “gluco-oligosaccharide” may also refer to an anhydro-glucose or a compound containing two or more glucose monosaccharide subunits linked by glycosidic bonds, wherein at least one monosaccharide subunit is replaced with an anhydro-glucose subunit. Similarly, a “galacto-oligosaccharide” may refer to a galactose or a compound containing two or more galactose monosaccharide subunits linked by glycosidic bonds. A “galacto-oligosaccharide” may also refer to an anhydro-galactose or a compound containing two or more galactose monosaccharide subunits linked by glycosidic bonds, wherein at least one monosaccharide subunit is replaced with an anhydro-galactose subunit. Analogously, a gluco-galactose-oligosaccharide may be a gluco-oligosaccharide, a galacto-oligosaccharide, or a compound containing one or more glucose monosaccharide subunits and one or more galactose monosaccharide subunits linked by glycosidic bonds, wherein at least one of the monosaccharide subunits is replaced with its respective anhydro-monosaccharide subunit. A gluco-galacto-xylo-oligosaccharide may refer to a compound produced by the condensation reaction of glucose, galactose, and xylose. An oligosaccharide preparation comprising gluco-galacto-xylo-oligosaccharides may comprise gluco-galactose-oligosaccharides, gluco-xylo-oligosaccharides, galacto-xylo-oligosaccharides, and compounds containing one or more glucose monosaccharide subunits, one or more xylose monosaccharide subunits, and one or more galactose monosaccharide subunits linked by glycosidic bonds.

As used herein, the term “monosaccharide unit” and “monosaccharide subunit” may be used interchangeably, unless suggested otherwise. A “monosaccharide subunit” may refer to a monosaccharide monomer in an oligosaccharide. For an oligosaccharide having a degree of polymerization of 1, the oligosaccharide may be referred to as a monosaccharide subunit or monosaccharide. For an oligosaccharide having a degree of polymerization higher than 1, its monosaccharide subunits are linked via glycosidic bonds.

As used herein, the term “regular monosaccharide” may refer to a monosaccharide that does not contain an anhydro-subunit. The term “regular disaccharide” may refer to a disaccharide that does not contain an anhydro-subunit. Accordingly, the term “regular subunit” may refer to a subunit that is not an anhydro-subunit.

The term “relative abundance” or “abundance,” as used herein, may refer to the abundance of a species in terms of how common or rare the species exists. For example, a DP1 fraction comprising 10% anhydro-subunit containing oligosaccharides by relative abundance may refer to a plurality of DP1 oligosaccharides, wherein 10%, by number, of the DP1 oligosaccharides are anhydro-monosaccharides.

Degree of Polymerization (DP) Distribution: A distribution of the degree of polymerization of the oligosaccharide preparation may be determined by any suitable analytical method and instrumentation, including but not limited to end group method, osmotic pressure (osmometry), ultracentrifugation, viscosity measurements, light scattering method, size exclusion chromatography (SEC), SEC-MALLS, field flow fractionation (FFF), asymmetric flow field flow fractionation (A4F), high-performance liquid chromatography (HPLC), and mass spectrometry (MS). For example, the distribution of the degree of polymerization may be determined and/or detected by mass spectrometry, such as MALDI-MS, LC-MS, or GC-MS. For another example, the distribution of the degree of polymerization may be determined and/or detected by SEC, such as gel permeation chromatography (GPC). As yet another example, the distribution of the degree of polymerization may be determined and/or detected by HPLC, FFF, or A4F. In another example, the degree of polymerization of the oligosaccharide preparation may be determined based on its molecular weight and molecular weight distribution (for a more detailed description see WO 2020/097458).

Anhydro-subunit Level: In some embodiments, each of the n fractions of oligosaccharides of the oligosaccharide preparation as described herein independently comprises an anhydro-subunit level. For instance, in some embodiments, the DP1 fraction comprises 10% anhydro-subunit containing oligosaccharides by relative abundance, and the DP2 fraction comprises 15% anhydro-subunit containing oligosaccharides by relative abundance. For another example, in some embodiments, DP1, DP2, and DP3 fraction each comprises 5%, 10%, and 2% anhydro-subunit containing oligosaccharides by relative abundance, respectively. In other embodiments, two or more fractions of oligosaccharides may comprise similar level of anhydro-subunit containing oligosaccharides. For example, in some embodiments, the DP1 and DP3 fraction each comprises about 5% anhydro-subunit containing oligosaccharides by relative abundance.

The level of anhydro-subunits may be determined by any suitable analytical methods, such as nuclear magnetic resonance (NMR) spectroscopy, mass spectrometry, HPLC, FFF, A4F, or any combination thereof. In some embodiments, the level of anhydro-subunits is determined, at least in part, by mass spectrometry such as MALDI-MS. In some embodiments, the level of anhydro-subunits may be determined, at least in part, by NMR. In some embodiments, the level of anhydro-subunits may be determined, at least in part, by HPLC. For example, in some embodiments, the level of anhydro-subunits may be determined by MALDI-MS, as illustrated in more detail in WO 2020/097458.

Glycosidic Linkages: In some embodiments, the oligosaccharide preparation described herein comprise a variety of glycosidic linkages. The type and distribution of the glycosidic linkages may depend on the source and manufacturing method of the oligosaccharide preparation. In some embodiments, the type and distribution of various glycosidic linkages may be determined and/or detected by any suitable methods known in the art such as NMR. For example, in some embodiments, the glycosidic linkages are determined and/or detected by proton NMR, carbon NMR, 2D NMR such as 2D JRES, HSQC, HMBC, DOSY, COSY, ECOSY, TOCSY, NOESY, or ROESY, or any combination thereof. In some embodiments, the glycosidic linkages are determined and/or detected, at least in part, by proton NMR. In some embodiments, the glycosidic linkages are determined and/or detected, at least in part, by carbon NMR. In some embodiments, the glycosidic linkages are determined and/or detected, at least in part, by 2D HSQC NMR.

In some embodiments, an oligosaccharide preparation may comprise one or more α-(1,2) glycosidic linkages, α-(1,3) glycosidic linkages, α-(1,4) glycosidic linkages, α-(1,6) glycosidic linkages, β-(1,2) glycosidic linkages, β-(1,3) glycosidic linkages, β-(1,4) glycosidic linkages, β-(1,6) glycosidic linkages, α(1,1)α glycosidic linkages, α(1,1)β glycosidic linkages, β(1,1)β glycosidic linkages, or any combination thereof.

In some embodiments, the oligosaccharide preparations have a glycosidic bond type distribution of about from 0 to 60 mol %, 5 to 55 mol %, 5 to 50 mol %, 5 to 45 mol %, 5 to 40 mol %, 5 to 35 mol %, 5 to 30 mol %, 5 to 25 mol %, 10 to 60 mol %, 10 to 55 mol %, 10 to 50 mol %, 10 to 45 mol %, 10 to 40 mol %, 10 to 35 mol %, 15 to 60 mol %, 15 to 55 mol %, 15 to 50 mol %, 15 to 45 mol %, 15 to 40 mol %, 15 to 35 mol %, 20 to 60 mol %, 20 to 55 mol %, 20 to 50 mol %, 20 to 45 mol %, 20 to 40 mol %, 20 to 35 mol %, 25 to 60 mol %, 25 to 55 mol %, 25 to 50 mol %, 25 to 45 mol %, 25 to 40 mol %, or 25 to 35 mol % of α-(1,6) glycosidic linkages.

Molecular Weight: The molecular weight and molecular weight distribution of the oligosaccharide preparation may be determined by any suitable analytical means and instrumentation, such as end group method, osmotic pressure (osmometry), ultracentrifugation, viscosity measurements, light scattering method, SEC, SEC-MALLS, FFF, A4F, HPLC, and mass spectrometry. In some embodiments, the molecular weight and molecular weight distribution are determined by mass spectrometry, such as MALDI-MS, LC-MS, or GC-MS. In some embodiments, the molecular weight and molecular weight distribution are determined by size exclusion chromatography (SEC), such as gel permeation chromatography (GPC). In other embodiments, the molecular weight and molecular weight distribution are determined by HPLC. In some embodiments, the molecular weight and molecular weight distribution are determined by MALDI-MS.

In some embodiments, the weight average molecular weight of the oligosaccharide preparation is about from 100 to 10000 g/mol, 200 to 8000 g/mol, 300 to 5000 g/mol, 500 to 5000 g/mol, 700 to 5000 g/mol, 900 to 5000 g/mol, 1100 to 5000 g/mol, 1300 to 5000 g/mol, 1500 to 5000 g/mol, 1700 to 5000 g/mol, 300 to 4500 g/mol, 500 to 4500 g/mol, 700 to 4500 g/mol, 900 to 4500 g/mol, 1100 to 4500 g/mol, 1300 to 4500 g/mol, 1500 to 4500 g/mol, 1700 to 4500 g/mol, 1900 to 4500 g/mol, 300 to 4000 g/mol, 500 to 4000 g/mol, 700 to 4000 g/mol, 900 to 4000 g/mol, 1100 to 4000 g/mol, 1300 to 4000 g/mol, 1500 to 4000 g/mol, 1700 to 4000 g/mol, 1900 to 4000 g/mol, 300 to 3000 g/mol, 500 to 3000 g/mol, 700 to 3000 g/mol, 900 to 3000 g/mol, 1100 to 3000 g/mol, 1300 to 3000 g/mol, 1500 to 3000 g/mol, 1700 to 3000 g/mol, 1900 to 3000 g/mol, 2100 to 3000 g/mol, 300 to 2500 g/mol, 500 to 2500 g/mol, 700 to 2500 g/mol, 900 to 2500 g/mol, 1100 to 2500 g/mol, 1300 to 2500 g/mol, 1500 to 2500 g/mol, 1700 to 2500 g/mol, 1900 to 2500 g/mol, 2100 to 2500 g/mol, 300 to 1500 g/mol, 500 to 1500 g/mol, 700 to 1500 g/mol, 900 to 1500 g/mol, 1100 to 1500 g/mol, 1300 to 1500 g/mol, 2000-2800 g/mol, 2100-2700 g/mol, 2200-2600 g/mol, 2300-2500 g/mol, or 2320-2420 g/mol. In some embodiments, the weight average molecular weight of the oligosaccharide preparation is about from 2000 to 2800 g/mol, 2100 to 2700 g/mol, 2200 to 2600 g/mol, 2300 to 2500 g/mol, or 2320 to 2420 g/mol.

Types of Olicosaccharides: In some embodiments, the species of oligosaccharides present in an oligosaccharide preparation referred to herein may depend on the type of the one or more feed sugars. For example, in some embodiments, the oligosaccharide preparations comprise a gluco-oligosaccharide when the feed sugars comprise glucose. For example, in some embodiments, the oligosaccharide preparations comprise a galacto-oligosaccharide when the feed sugars comprise galactose. For another example, in some embodiments, the oligosaccharide preparations comprise gluco-galacto-oligosaccharides when the feed sugars comprise galactose and glucose.

In some embodiments, the oligosaccharide preparations comprise one or more species of monosaccharide subunits. In some embodiments, the oligosaccharide preparation may comprise oligosaccharides with 1, 2, 3, 4, 5, 6, 7, 8, 9, 10, 11, 12, 13, 14, 15, 16, 17, 18, 19, 20, or more different species of monosaccharides subunits.

Method of Manufacturing Oligosaccharide Preparations: The Method of manufacturing an oligosaccharide preparation according to the invention is described in detail in WO 2020/097458 comprising heating an aqueous composition comprising one or more feed sugars and a catalyst to a temperature and for a time sufficient to induce polymerization, wherein the catalyst is selected from the group consisting of: (+)-camphor-10-sulfonic acid; 2-pyridinesulfonic acid; 3-pyridinesulfonic acid; 8-hydroxy-5-quinolinesulfonic acid hydrate; α-hydroxy-2-pyridinemethanesulfonic acid; (β)-camphor-10-sulfonic acid; butylphosphonic acid; diphenylphosphinic acid; hexylphosphonic acid; methylphosphonic acid; phenylphosphinic acid; phenylphosphonic acid; tert-butylphosphonic acid; SS)-VAPOL hydrogenphosphate; 6-quinolinesulfonic acid, 3-(1-pyridinio)-1-propanesulfonate; 2-(2-pyridinyl)ethanesulfonic acid; 3-(2-pyridyl)-5,6-diphenyl-1,2,4-triazine-p,p′-disulfonic acid monosodium salt hydrate; 1,1′-binaphthyl-2,2′-diyl-hydrogenphosphate; bis(4-methoxyphenyl)phosphinic acid; phenyl(3,5-xylyl)phosphinic acid; L-cysteic acid monohydrate; poly(styrene sulfonic acid-co-divinylbenzene); lysine; Ethanedisulfonic acid; Ethanesulfonic acid; Isethionic acid; Homocysteic acid; HEPBS (N-(2-Hydroxyethyl)piperazine-N′-(4-butanesulfonic acid)); HEPES (4-(2-hydroxyethyl)-1-piperazineethanesulfonic acid); 2-Hydroxy-3-morpholinopropanesulfonic acid; 2-(N-morpholino)ethanesulfonic acid; Methanesulfonic acid; Methaniazide; Naphthalene-1-sulfonic acid; Naphthalene-2-sulfonic acid; Perfluorobutanesulfonic acid; 6-sulfoquinovose; Triflic acid; 2-aminoethanesulfonic acid; Benzoic acid; Chloroacetic acid; Trifluoroacetic acid; Caproic acid; Enanthic acid; Caprylic acid; Pelargonic acid; Lauric acid; Pamitic acid; Stearic acid; Arachidic acid; Aspartic acid; Glutamic acid; Serine; Threonine; Glutamine; Cysteine; Glycine; Proline; Alanine; Valine; Isoleucine; Leucine; Methionine; Phenylalanine; Tyrosine; Tryptophan.

In some embodiments, the polymerization of the feed sugars is achieved by a step-growth polymerization. In some embodiments, the polymerization of the feed sugars is achieved by polycondensation.

Feed Sugar: The one or more feed sugars used in the methods of manufacturing oligosaccharide preparations described herein may comprise one or more types of sugars. In some embodiments, the one or more feed sugars comprise monosaccharides, disaccharides, trisaccharides, tetrasaccharides, or any mixtures thereof.

In some embodiments, the one or more feed sugars comprise glucose. In some embodiments, the one or more feed sugars comprise glucose and galactose. In some embodiments, the one or more feed sugars comprise glucose, xylose, and galactose. In some embodiments, the one or more feed sugars comprise glucose and mannose. In some embodiments, the one or more feed sugars comprise glucose and fructose. In some embodiments, the one or more feed sugars comprise glucose, fructose, and galactose. In some embodiments, the one or more feed sugars comprise glucose, galactose, and mannose.

As used herein, the singular forms “a,” “and,” and “the” include plural referents unless the context clearly dictates otherwise. Thus, for example, reference to “an agent” includes a plurality of such agents, and reference to “the oligosaccharide” includes reference to one or more oligosaccharides (or to a plurality of oligosaccharides) and equivalents thereof known to those skilled in the art, and so forth.

When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range.

The invention is further characterized by the following items:
Item 1. Use of an oligosaccharide preparation (e.g. a synthetic oligosaccharide preparation) to increase average body weight and/or to decrease feed conversion ratio and/or to increase average body weight gain of a bird vaccinated against coccidiosis, wherein the oligosaccharide preparation comprises at least n fractions of oligosaccharides each having a distinct degree of polymerization selected from 1 to n (DP1 to DPn fractions), wherein n is an integer greater than or equal to 2;
wherein each fraction comprises from at least about 0.5% to about 90% (e.g. from 1% to 90%; from about 0.5% to about 15%) of anhydro-subunit containing oligosaccharides by relative abundance as determined by mass spectrometry; and
wherein the average body weight of the bird is increased in comparison to a comparable control bird; and/or the feed conversion ratio of the bird is decreased in comparison to the comparable control bird; and/or the average body weight gain of the bird is increased in comparison to the comparable control bird; wherein the comparable control bird was not administered the oligosaccharide preparation.
Item 2. Use according to item 1, wherein the average body weight of the bird is increased and/or the feed conversion ratio of the bird is decreased and/or the average body weight gain of the bird is increased on at least one day within 42 days after vaccination against coccidiosis (e.g. on one or more day(s) selected from the group of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42 day(s) after vaccination against coccidiosis).
Item 3. Use according to item 2, wherein the average body weight is increased by at least 0.2% (e.g. at least 0.5%, 0.75%, 1.00%, 1.25%, 1.50%, 1.68%, 1.70%, 2.0%, 2.5%, 2.7% etc.); and/or wherein the feed conversion ratio is decreased by at least 0.2% (e.g. at least 0.5%, 0.75%, 1.00%, 1.25%, 1.50%, 1.7%, 2.0%, 2.4%, 2.5%, 2.7%, 2.8% etc.); and/or wherein the average body weight gain is increased by at least 0.2% (e.g. at least 0.5%, 0.75%, 1.0%, 1.5%, 2%, 2.5%, 2.7%, 2.8%, 3.0%) on at least one day within 42 days after vaccination against coccidiosis (e.g. on one or more day(s) selected from the group of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42 day(s) after vaccination against coccidiosis).
Item 4. Use of an oligosaccharide preparation (e.g. synthetic oligosaccharide preparation) to adapt the average body weight and/or the feed conversion ratio and/or the average body weight gain of a bird vaccinated against coccidiosis to match the average body weight and/or the feed conversion ratio and/or the average body weight gain of a bird not vaccinated against coccidiosis;
wherein the oligosaccharide preparation comprises at least n fractions of oligosaccharides each having a distinct degree of polymerization selected from 1 to n (DP1 to DPn fractions), wherein n is an integer greater than or equal to 2; and
wherein each fraction comprises from at least about 0.5% to about 90% (e.g. from 1% to 90%; from about 0.5% to about 15%) of anhydro-subunit containing oligosaccharides by relative abundance as determined by mass spectrometry.
Item 5. Use according to item 4, wherein the average body weight and/or the feed conversion ratio and/or the average body weight gain of the bird vaccinated against coccidiosis matches the average body weight and/or the feed conversion ratio and/or the average body weight gain of a bird vaccinated against coccidiosis of the bird not vaccinated against coccidiosis on at least one day within 42 days after vaccination against coccidiosis (e.g. on one or more day(s) selected from the group of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42 day(s) after vaccination against coccidiosis).
Item 6. Use according to any one of items 4 and 5, wherein the average body weight of the bird not vaccinated against coccidiosis is at least 0.2% (e.g. at least 0.5%, 0.75%, 1.00%, 1.25%, 1.50%, 1.68%, 1.70%, 2.0%, 2.5%, 2.7% etc.) higher than the average body weight of a bird vaccinated against coccidiosis and not administered the oligosaccharide preparation; and or the feed conversion ratio of the bird not vaccinated against coccidiosis is at least 0.2% (e.g. at least 0.5%, 0.75%, 1.00%, 1.25%, 1.50%, 1.7%, 2.0%, 2.4%, 2.5%, 2.7%, 2.8% etc.) lower than the feed conversion ratio of a bird vaccinated against coccidiosis and not administered the oligosaccharide preparation; and/or the average body weight gain of a bird not vaccinated against coccidiosis is at least 0.2% (e.g. at least 0.5%, 0.75%, 1.0%, 1.5%, 2%, 2.5%, 2.7%, 2.8%, 3.0%) higher than the average body weight gain of a bird vaccinated against coccidiosis and not administered the oligosaccharide preparation
on at least one day within 42 days after vaccination against coccidiosis (e.g. on one or more day(s) selected from the group of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42 day(s) after vaccination against coccidiosis).
Item 7. Use according to any one of the preceding items, wherein the oligosaccharide preparation is comprised in a nutritional composition administered to the bird.
Item 8. Use according to claim 7, wherein the oligosaccharide preparation is comprised in the nutritional composition at an inclusion rate of at least 50 ppm (e.g. at least 50, 70, 100, 150, 200, 300, 400, 500 ppm).
Item 9. Use according to any one of the preceding items, wherein the bird is poultry (e.g. chicken, duck, goose, turkey, guinea fowl, pigeon, quail), preferably chicken.
Item 10. Use according to any one of the preceding items, wherein n is at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100.
Item 11. Use according to any one of the preceding items, wherein at least one fraction of the oligosaccharide preparation comprises less than 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, or 2% anhydro-subunit containing oligosaccharides by relative abundance.
Item 12. Use according to any one of the preceding items, wherein at least one fraction of the oligosaccharide preparation comprises less than 10%, 9%, 8%, 7%, 6%, 5%, 4%, 3%, or 2% anhydro-subunit containing oligosaccharides by relative abundance.
Item 13. Use according to any one of the preceding items, wherein each fraction of the oligosaccharide preparation comprises greater than 0.2%, 0.5%, 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, or 80% anhydro-subunit containing oligosaccharides by relative abundance.
Item 14. Use according to any one of the preceding items, wherein the oligosaccharide preparation has a weight average molecular weight from about 300 to 5000 g/mol, 500 to 5000 g/mol, 700 to 5000 g/mol, 500 to 2000 g/mol, 700 to 2000 g/mol, 700 to 1500 g/mol, 300 to 1500 g/mol, 300 to 2000 g/mol, 400 to 1300 g/mol, 400 to 1200 g/mol, 400 to 1100 g/mol, 500 to 1300 g/mol, 500 to 1200 g/mol, 500 to 1100 g/mol, 600 to 1300 g/mol, 600 to 1200 g/mol, or 600 to 1100 g/mol.
Item 15. Use according to any one of the preceding items, wherein the oligosaccharide preparation has a weight average molecular weight from about 2000 to 2800 g/mol, 2100 to 2700 g/mol, 2200 to 2600 g/mol, 2300 to 2500 g/mol, or 2320 to 2420 g/mol.
Item 16. Use according to any one of the preceding items, wherein the oligosaccharide preparation has a number average molecular weight from about 1000 to 2000 g/mol, 1100 to 1900 g/mol, 1200 to 1800 g/mol, 1300 to 1700 g/mol, 1400 to 1600 g/mol, or 1450 to 1550 g/mol.
Item 17. Use according to any one of the preceding items, wherein the oligosaccharide preparation is administered to the bird before, after, and/or simultaneously with the diet of the bird vaccinated against coccidiosis, preferably simultaneously with the diet.
Item 18: Use according to any one of the preceding items, wherein the relative abundance of oligosaccharides in each of the n fractions of the oligosaccharide preparation decreases monotonically with its degree of polymerization.
Item 19: Use according to any one of the preceding items, wherein the relative abundance of oligosaccharides in at least 5, 10, 20, or 30 DP fractions of the oligosaccharide preparation decreases monotonically with its degree of polymerization.
Item 20: Use according to any one of the preceding items, wherein at least one fraction of the oligosaccharide preparation comprises less than 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, or 2% anhydro-subunit containing oligosaccharides by relative abundance.
Item 21: Use according to any one of the preceding items, wherein the oligosaccharide preparation comprises less than 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, or 2% anhydro-subunit containing oligosaccharides by relative abundance.
Item 22: Use according to any one of the preceding items, wherein each fraction of the oligosaccharide preparation comprises less than 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, or 2% anhydro-subunit containing oligosaccharides by relative abundance.
Item 23: Use according to any one of the preceding items, wherein at least one fraction of the oligosaccharide preparation comprises less than 10%, 9%, 8%, 7%, 6%, 5%, 4%, 3%, or 2% anhydro-subunit containing oligosaccharides by relative abundance.
Item 24: Use according to any one of the preceding items, wherein the oligosaccharide preparation comprises less than 10%, 9%, 8%, 7%, 6%, 5%, 4%, 3%, or 2% anhydro-subunit containing oligosaccharides by relative abundance.
Item 25: Use according to any one of the preceding items, wherein each fraction of the oligosaccharide preparation comprises less than 10%, 9%, 8%, 7%, 6%, 5%, 4%, 3%, or 2% anhydro-subunit containing oligosaccharides by relative abundance.
Item 26: Use according to any one of the preceding items, wherein at least one fraction of the oligosaccharide preparation comprises greater than 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, or 80% anhydro-subunit containing oligosaccharides by relative abundance.
Item 27: Use according to any one of the preceding items, wherein the oligosaccharide preparation comprises greater than 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, or 80% anhydro-subunit containing oligosaccharides by relative abundance.
Item 28: Use according to any one of the preceding items, wherein each fraction of the oligosaccharide preparation comprises greater than 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, or 80% anhydro-subunit containing oligosaccharides by relative abundance.
Item 29: Use according to any one of the preceding items, wherein at least one fraction of the oligosaccharide preparation comprises greater than 20%, 21%, 22%, 23%, 24%, or 25% anhydro-subunit containing oligosaccharides by relative abundance.
Item 30: Use according to any one of the preceding items, wherein the oligosaccharide preparation comprises greater than 20%, 21%, 22%, 23%, 24%, or 25% anhydro-subunit containing oligosaccharides by relative abundance.
Item 31: Use according to any one of the preceding items, wherein each fraction of the oligosaccharide preparation comprises greater than 20%, 21%, 22%, 23%, 24%, or 25% anhydro-subunit containing oligosaccharides by relative abundance.
Item 32: Use according to any one of the preceding items, wherein more than 99%, 95%, 90%, 85%, 80%, 75%, 70%, 65%, 60%, 55%, 50%, 45%, 40%, 35%, or 30% of the anhydro-subunit containing oligosaccharides of the oligosaccharide preparation have only one anhydro-subunit.
Item 33: Use according to any one of the preceding items, wherein the oligosaccharide preparation has a DP1 fraction content from 1 to 40% by relative abundance.
Item 34: Use according to any one of the preceding items, wherein the oligosaccharide preparation has a DP2 fraction content from 1 to 35% by relative abundance.
Item 35: Use according to any one of the preceding items, wherein the oligosaccharide preparation has a DP3 fraction content from 1 to 30% by relative abundance.
Item 36: Use according to any one of the preceding items, wherein the oligosaccharide preparation has a DP4 fraction content from 0.1 to 20% by relative abundance.
Item 37: Use according to any one of the preceding items, wherein the oligosaccharide preparation has a DP5 fraction content from 0.1 to 15% by relative abundance.
Item 38: Use according to any one of the preceding items, wherein the ratio of DP2 fraction to DP1 fraction of the oligosaccharide preparation is 0.02-0.40 by relative abundance.
Item 39: Use according to any one of the preceding items, wherein the ratio of DP3 fraction to DP2 fraction of the oligosaccharide preparation is 0.01-0.30 by relative abundance.
Item 40: Use according to any one of the preceding items, wherein the aggregate content of DP1 and DP2 fractions in the oligosaccharide preparation is less than 50, 30, or 10% by relative abundance.
Item 41: Use according to any one of the preceding items, wherein the oligosaccharide preparation comprises at least 103, 104, 105, 106 or 109 different oligosaccharide species.
Item 42: Use according to any one of the preceding items, wherein two or more independent oligosaccharides of the oligosaccharide preparation comprise different anhydro-subunits.
Item 43: Use according to any one of the preceding items, wherein the oligosaccharide preparation comprises one or more anhydro-subunits that are products of reversible thermal dehydration of monosaccharides.
Item 44: Use according to any one of the preceding items, wherein the oligosaccharide preparation comprises one or more anhydro-glucose, anhydro-galactose, anhydro-mannose, anhydro-allose, anhydro-altrose, anhydro-gulose, anhydro-indose, anhydro-talose, anhydro-fructose, anhydro-ribose, anhydro-arabinose, anhydro-rhamnose, anhydro-lyxose, or anhydro-xylose subunits.
Item 45: Use according to any one of the preceding items, wherein the oligosaccharide preparation comprises one or more anhydro-glucose, anhydro-galactose, anhydro-mannose, or anhydro-fructose subunits.
Item 46: Use according to any one of the preceding items, wherein the oligosaccharide preparation comprises one or more 1,6-anhydro-β-D-glucofuranose or 1,6-anhydro-β-D-glucopyranose subunits. In some embodiments, the oligosaccharide preparation comprises both 1,6-anhydro-β-D-glucofuranose and 1,6-anhydro-β-D-glucopyranose anhydro-subunits.
Item 47: Use according to any one of the preceding items, wherein a ratio of 1,6-anhydro-β-D-glucofuranose to 1,6-anhydro-β-D-glucopyranose is from about 10:1 to 1:10, 9:1 to 1:10, 8:1 to 1:10, 7:1 to 1:10, 6:1 to 1:10, 5:1 to 1:10, 4:1 to 1:10, 3:1 to 1:10, 2:1 to 1:10, 10:1 to 1:9, 10:1 to 1:8, 10:1 to 1:7, 10:1 to 1:6, 10:1 to 1:5, 10:1 to 1:4, 10:1 to 1:3, 10:1 to 1:2, or 1:1 to 3:1 in the oligosaccharide preparation.
Item 48: Use according to any one of the preceding items, wherein the ratio of 1,6-anhydro-β-D-glucofuranose to 1,6-anhydro-β-D-glucopyranose is about 10:1, 9:1, 8:1, 7:1, 6:1, 5:1, 4:1, 3:1, 2:1, 1:1, 1:2, 1:3, 1:4, 1:5, 1:6, 1:7, 1:8, 1:8, 1:9, or 1:10 within the oligosaccharide preparation.
Item 49: Use according to any one of the preceding items, wherein the ratio of 1,6-anhydro-β-D-glucofuranose to 1,6-anhydro-β-D-glucopyranose is about 2:1 in the oligosaccharide preparation.
Item 50: Use according to any one of the preceding items, wherein the ratio of 1,6-anhydro-β-D-glucofuranose to 1,6-anhydro-β-D-glucopyranose is about from 10:1 to 1:10, 9:1 to 1:10, 8:1 to 1:10, 7:1 to 1:10, 6:1 to 1:10, 5:1 to 1:10, 4:1 to 1:10, 3:1 to 1:10, 2:1 to 1:10, 10:1 to 1:9, 10:1 to 1:8, 10:1 to 1:7, 10:1 to 1:6, 10:1 to 1:5, 10:1 to 1:4, 10:1 to 1:3, 10:1 to 1:2, or 1:1 to 3:1 in each fraction of the oligosaccharide preparation.
Item 51: Use according to any one of the preceding items, wherein the ratio of 1,6-anhydro-β-D-glucofuranose to 1,6-anhydro-β-D-glucopyranose is about 10:1, 9:1, 8:1, 7:1, 6:1, 5:1, 4:1, 3:1, 2:1, 1:1, 1:2, 1:3, 1:4, 1:5, 1:6, 1:7, 1:8, 1:8, 1:9, or 1:10 in each fraction of the oligosaccharide preparation.
Item 52: Use according to any one of the preceding items, wherein the ratio of 1,6-anhydro-β-D-glucofuranose to 1,6-anhydro-β-D-glucopyranose is about 2:1 in each fraction of the oligosaccharide preparation.
Item 53: Use according to anyone of the preceding items, wherein at least 1%, 5%, 10%, 20%, 30%, 40%, 50%, 60%, 70%, 80%, or 90% of anhydro-subunits in the oligosaccharide preparation are selected from a group consisting of 1,6-anhydro-β-D-glucofuranose and 1,6-anhydro-s-D-glucopyranose.
Item 54: Use according to any one of the preceding items, wherein the weight average molecular weight of the oligosaccharide preparation is about from 300 to 5000 g/mol, 500 to 5000 g/mol, 700 to 5000 g/mol, 500 to 2000 g/mol, 700 to 2000 g/mol, 700 to 1500 g/mol, 300 to 1500 g/mol, 300 to 2000 g/mol, 400 to 1300 g/mol, 400 to 1200 g/mol, 400 to 1100 g/mol, 500 to 1300 g/mol, 500 to 1200 g/mol, 500 to 1100 g/mol, 600 to 1300 g/mol, 600 to 1200 g/mol, or 600 to 1100 g/mol.
Item 55: Use according to any one of the preceding items, wherein the number average molecular weight of the oligosaccharide preparation is about from 300 to 5000 g/mol, 500 to 5000 g/mol, 700 to 5000 g/mol, 500 to 2000 g/mol, 700 to 2000 g/mol, 700 to 1500 g/mol, 300 to 1500 g/mol, 300 to 2000 g/mol, 400 to 1000 g/mol, 400 to 900 g/mol, 400 to 800 g/mol, 500 to 900 g/mol, or 500 to 800 g/mol.
Item 56: Use according to any one of the preceding items, wherein the distribution of the degree of polymerization is determined and/or detected by MALDI-MS, GC-MS, LC-MS, SEC, HPLC and/or combination(s) thereof (e.g. MALDI-MS and SEC).
Item 58: Use according to any one of the preceding items, wherein the degree of polymerization of the oligosaccharide preparation may be determined based on its molecular weight and molecular weight distribution.
Item 59. Oligosaccharide preparation (e.g. synthetic oligosaccharide preparation) for use in treatment, amelioration and/or prophylaxis of a bird vaccinated against coccidiosis, wherein the oligosaccharide preparation comprises at least n fractions of oligosaccharides each having a distinct degree of polymerization selected from 1 to n (DP1 to DPn fractions), wherein n is an integer greater or equal to 2; and wherein each fraction comprises from at least about 0.5% to about 90% (e.g. from 1% to 90%) of anhydro-subunit containing oligosaccharides by relative abundance as determined by mass spectrometry.
Item 60. Oligosaccharide preparation for use in treatment, amelioration and/or prophylaxis according to item 59, wherein the treatment, amelioration and/or prophylaxis comprises the step of administering the oligosaccharide preparation to the bird from day of vaccination against coccidiosis for at least 1 day (e.g. for at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 37, 39, 40, 41, 42 days).
Item 61. Oligosaccharide preparation (e.g. synthetic oligosaccharide preparation) for use in treatment, amelioration and/or prophylaxis of at least one adverse effect of vaccination against coccidiosis in a bird vaccinated against coccidiosis, wherein the oligosaccharide preparation comprises at least n fractions of oligosaccharides each having a distinct degree of polymerization selected from 1 to n (DP1 to DPn fractions), wherein n is an integer greater or equal to 2; and wherein each fraction comprises from at least about 0.5% to about 90% (e.g. from 1% to 90%) of anhydro-subunit containing oligosaccharides by relative abundance as determined by mass spectrometry.
Item 62. Oligosaccharide preparation for use in treatment, amelioration and/or prophylaxis according to item 61, wherein the treatment, amelioration and/or prophylaxis comprises the step of administering the oligosaccharide preparation to the bird from day of vaccination against coccidiosis for at least 1 day (e.g. for at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 37, 39, 40, 41, 42 days).
Item 63. Oligosaccharide preparation for use in treatment, amelioration and/or prophylaxis according to any one of items 59-62, wherein the oligosaccharide preparation is administered to the bird before, after, and/or simultaneously with the diet of the bird vaccinated against coccidiosis, preferably simultaneously with the diet.
Item 64. Oligosaccharide preparation for use in treatment, amelioration and/or prophylaxis according to any one of items 59-63, wherein the oligosaccharide preparation is comprised in a nutritional composition administered to the bird.
Item 65: Oligosaccharide preparation for use in treatment, amelioration and/or prophylaxis according to item 64, wherein the oligosaccharide preparation is comprised in the nutritional composition at an inclusion rate of at least 50 ppm (e.g. at least 50, 70, 100, 150, 200, 300, 400, 500 ppm).
Item 66: Oligosaccharide preparation for use in treatment, amelioration and/or prophylaxis according to any one of items 59-65, wherein the bird is poultry (e.g. chicken, duck, goose, turkey, guinea fowl, pigeon, quail), preferably chicken.
Item 67: Oligosaccharide preparation for use in treatment, amelioration and/or prophylaxis according to any one of items 59-66, wherein n is at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100.
Item 68. Oligosaccharide preparation for use in treatment, amelioration and/or prophylaxis according to any one of items 59-67, wherein at least one fraction of the oligosaccharide preparation comprises less than 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, or 2% anhydro-subunit containing oligosaccharides by relative abundance.
Item 69. Oligosaccharide preparation for use in treatment, amelioration and/or prophylaxis according to any one of items 59-68, wherein at least one fraction of the oligosaccharide preparation comprises less than 10%, 9%, 8%, 7%, 6%, 5%, 4%, 3%, or 2% anhydro-subunit containing oligosaccharides by relative abundance.
Item 70. Oligosaccharide preparation for use in treatment, amelioration and/or prophylaxis according to any one of items 59-69, wherein each fraction of the oligosaccharide preparation comprises greater than 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, or 80% anhydro-subunit containing oligosaccharides by relative abundance.
Item 71. Oligosaccharide preparation for use in treatment, amelioration and/or prophylaxis according to any one of items 59-70, wherein the oligosaccharide preparation has a weight average molecular weight from about 300 to 5000 g/mol, 500 to 5000 g/mol, 700 to 5000 g/mol, 500 to 2000 g/mol, 700 to 2000 g/mol, 700 to 1500 g/mol, 300 to 1500 g/mol, 300 to 2000 g/mol, 400 to 1300 g/mol, 400 to 1200 g/mol, 400 to 1100 g/mol, 500 to 1300 g/mol, 500 to 1200 g/mol, 500 to 1100 g/mol, 600 to 1300 g/mol, 600 to 1200 g/mol, or 600 to 1100 g/mol.
Item 72. Oligosaccharide preparation for use in treatment, amelioration and/or prophylaxis according to any one of items 59-71, wherein the oligosaccharide preparation has a weight average molecular weight from about 2000 to 2800 g/mol, 2100 to 2700 g/mol, 2200 to 2600 g/mol, 2300 to 2500 g/mol, or 2320 to 2420 g/mol.
Item 73. Oligosaccharide preparation for use in treatment, amelioration and/or prophylaxis according to any one of items 59-72, wherein the oligosaccharide preparation has a number average molecular weight from about 1000 to 2000 g/mol, 1100 to 1900 g/mol, 1200 to 1800 g/mol, 1300 to 1700 g/mol, 1400 to 1600 g/mol, or 1450 to 1550 g/mol.
Item 74: Oligosaccharide preparation for use in treatment, amelioration and/or prophylaxis according to any one of items 59-73, wherein the relative abundance of oligosaccharides in each fraction of the oligosaccharide preparation decreases monotonically with its degree of polymerization.
Item 75: Oligosaccharide preparation for use in treatment, amelioration and/or prophylaxis according to any one of items 59-74, wherein the relative abundance of oligosaccharides in at least 5, 10, 20, or 30 DP fractions of the oligosaccharide preparation decreases monotonically with its degree of polymerization.
Item 76: Oligosaccharide preparation for use in treatment, amelioration and/or prophylaxis according to any one of items 59-75, wherein at least one fraction of the oligosaccharide preparation comprises less than 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, or 2% anhydro-subunit containing oligosaccharides by relative abundance.
Item 77: Oligosaccharide preparation for use in treatment, amelioration and/or prophylaxis according to any one of items 59-76, wherein the oligosaccharide preparation comprises less than 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, or 2% anhydro-subunit containing oligosaccharides by relative abundance.
Item 78: Oligosaccharide preparation for use in treatment, amelioration and/or prophylaxis according to any one of items 59-77, wherein each fraction of the oligosaccharide preparation comprises less than 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, or 2% anhydro-subunit containing oligosaccharides by relative abundance.
Item 79: Oligosaccharide preparation for use in treatment, amelioration and/or prophylaxis according to any one of items 59-78, wherein at least one fraction of the oligosaccharide preparation comprises less than 10%, 9%, 8%, 7%, 6%, 5%, 4%, 3%, or 2% anhydro-subunit containing oligosaccharides by relative abundance.
Item 80: Oligosaccharide preparation for use in treatment, amelioration and/or prophylaxis according to any one of items 59-79, wherein the oligosaccharide preparation comprises less than 10%, 9%, 8%, 7%, 6%, 5%, 4%, 3%, or 2% anhydro-subunit containing oligosaccharides by relative abundance.
Item 81: Oligosaccharide preparation for use in treatment, amelioration and/or prophylaxis according to any one of items 59-80, wherein each fraction of the oligosaccharide preparation comprises less than 10%, 9%, 8%, 7%, 6%, 5%, 4%, 3%, or 2% anhydro-subunit containing oligosaccharides by relative abundance.
Item 82: Oligosaccharide preparation for use in treatment, amelioration and/or prophylaxis according to any one of items 59-81, wherein at least one fraction of the oligosaccharide preparation comprises greater than 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, or 80% anhydro-subunit containing oligosaccharides by relative abundance.
Item 83: Oligosaccharide preparation for use in treatment, amelioration and/or prophylaxis according to any one of items 59-82, wherein the oligosaccharide preparation comprises greater than 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, or 80% anhydro-subunit containing oligosaccharides by relative abundance.
Item 84: Oligosaccharide preparation for use in treatment, amelioration and/or prophylaxis according to any one of items 59-83, wherein each fraction of the oligosaccharide preparation comprises greater than 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, or 80% anhydro-subunit containing oligosaccharides by relative abundance.
Item 85: Oligosaccharide preparation for use in treatment, amelioration and/or prophylaxis according to any one of items 59-84, wherein at least one fraction of the oligosaccharide preparation comprises greater than 20%, 21%, 22%, 23%, 24%, or 25% anhydro-subunit containing oligosaccharides by relative abundance.
Item 86: Oligosaccharide preparation for use in treatment, amelioration and/or prophylaxis according to any one of items 59-85, wherein the oligosaccharide preparation comprises greater than 20%, 21%, 22%, 23%, 24%, or 25% anhydro-subunit containing oligosaccharides by relative abundance.
Item 87: Oligosaccharide preparation for use in treatment, amelioration and/or prophylaxis according to any one of items 59-86, wherein each fraction of the oligosaccharide preparation comprises greater than 20%, 21%, 22%, 23%, 24%, or 25% anhydro-subunit containing oligosaccharides by relative abundance.
Item 88: Oligosaccharide preparation for use in treatment, amelioration and/or prophylaxis according to any one of items 59-87, wherein more than 99%, 95%, 90%, 85%, 80%, 75%, 70%, 65%, 60%, 55%, 50%, 45%, 40%, 35%, or 30% of the anhydro-subunit containing oligosaccharides of the oligosaccharide preparation have only one anhydro-subunit.
Item 89: Oligosaccharide preparation for use in treatment, amelioration and/or prophylaxis according to any one of items 59-88, wherein the oligosaccharide preparation has a DP1 fraction content from 1 to 40% by relative abundance.
Item 90: Oligosaccharide preparation for use in treatment, amelioration and/or prophylaxis according to any one of items 59-89, wherein the oligosaccharide preparation has a DP2 fraction content from 1 to 35% by relative abundance.
Item 91: Oligosaccharide preparation for use in treatment, amelioration and/or prophylaxis according to any one of items 59-90, wherein the oligosaccharide preparation has a DP3 fraction content from 1 to 30% by relative abundance.
Item 92: Oligosaccharide preparation for use in treatment, amelioration and/or prophylaxis according to any one of items 59-91, wherein the oligosaccharide preparation has a DP4 fraction content from 0.1 to 20% by relative abundance.
Item 93: Oligosaccharide preparation for use in treatment, amelioration and/or prophylaxis according to any one of items 59-92, wherein the oligosaccharide preparation has a DP5 fraction content from 0.1 to 15% by relative abundance.
Item 94: Oligosaccharide preparation for use in treatment, amelioration and/or prophylaxis according to any one of items 59-93, wherein the ratio of DP2 fraction to DP1 fraction of the oligosaccharide preparation is 0.02-0.40 by relative abundance.
Item 95: Oligosaccharide preparation for use in treatment, amelioration and/or prophylaxis according to any one of items 59-94, wherein the ratio of DP3 fraction to DP2 fraction of the oligosaccharide preparation is 0.01-0.30 by relative abundance.
Item 96: Oligosaccharide preparation for use in treatment, amelioration and/or prophylaxis according to any one of items 59-95, wherein the aggregate content of DP1 and DP2 fractions in the oligosaccharide preparation is less than 50, 30, or 10% by relative abundance.
Item 97: Oligosaccharide preparation for use in treatment, amelioration and/or prophylaxis according to any one of items 59-96, wherein the oligosaccharide preparation comprises at least 103, 104, 105, 106 or 109 different oligosaccharide species.
Item 98: Oligosaccharide preparation for use in treatment, amelioration and/or prophylaxis according to any one of items 59-97, wherein two or more independent oligosaccharides of the oligosaccharide preparation comprise different anhydro-subunits.
Item 99: Oligosaccharide preparation for use in treatment, amelioration and/or prophylaxis according to any one of items 59-98, wherein the oligosaccharide preparation comprises one or more anhydro-subunits that are products of reversible thermal dehydration of monosaccharides.
Item 100: Oligosaccharide preparation for use in treatment, amelioration and/or prophylaxis according to any one of items 59-99, wherein the oligosaccharide preparation comprises one or more anhydro-glucose, anhydro-galactose, anhydro-mannose, anhydro-allose, anhydro-altrose, anhydro-gulose, anhydro-indose, anhydro-talose, anhydro-fructose, anhydro-ribose, anhydro-arabinose, anhydro-rhamnose, anhydro-lyxose, or anhydro-xylose subunits.
Item 101: Oligosaccharide preparation for use in treatment, amelioration and/or prophylaxis according to any one of items 59-100, wherein the oligosaccharide preparation comprises one or more anhydro-glucose, anhydro-galactose, anhydro-mannose, or anhydro-fructose subunits.
Item 102: Oligosaccharide preparation for use in treatment, amelioration and/or prophylaxis according to any one of items 59-101, wherein the oligosaccharide preparation comprises one or more 1,6-anhydro-β-D-glucofuranose or 1,6-anhydro-β-D-glucopyranose subunits. In some embodiments, the oligosaccharide preparation comprises both 1,6-anhydro-β-D-glucofuranose and 1,6-anhydro-β-D-glucopyranose anhydro-subunits.
Item 103: Oligosaccharide preparation for use in treatment, amelioration and/or prophylaxis according to any one of items 59-102, wherein a ratio of 1,6-anhydro-β-D-glucofuranose to 1,6-anhydro-β-D-glucopyranose is from about 10:1 to 1:10, 9:1 to 1:10, 8:1 to 1:10, 7:1 to 1:10, 6:1 to 1:10, 5:1 to 1:10, 4:1 to 1:10, 3:1 to 1:10, 2:1 to 1:10, 10:1 to 1:9, 10:1 to 1:8, 10:1 to 1:7, 10:1 to 1:6, 10:1 to 1:5, 10:1 to 1:4, 10:1 to 1:3, 10:1 to 1:2, or 1:1 to 3:1 in the oligosaccharide preparation.
Item 104: Oligosaccharide preparation for use in treatment, amelioration and/or prophylaxis according to any one of items 59-103, wherein the ratio of 1,6-anhydro-β-D-glucofuranose to 1,6-anhydro-β-D-glucopyranose is about 10:1, 9:1, 8:1, 7:1, 6:1, 5:1, 4:1, 3:1, 2:1, 1:1, 1:2, 1:3, 1:4, 1:5, 1:6, 1:7, 1:8, 1:8, 1:9, or 1:10 within the oligosaccharide preparation.
Item 105: Oligosaccharide preparation for use in treatment, amelioration and/or prophylaxis according to any one of items 59-104, wherein the ratio of 1,6-anhydro-β-D-glucofuranose to 1,6-anhydro-β-D-glucopyranose is about 2:1 in the oligosaccharide preparation.
Item 106: Oligosaccharide preparation for use in treatment, amelioration and/or prophylaxis according to any one of items 59-105, wherein the ratio of 1,6-anhydro-β-D-glucofuranose to 1,6-anhydro-β-D-glucopyranose is about from 10:1 to 1:10, 9:1 to 1:10, 8:1 to 1:10, 7:1 to 1:10, 6:1 to 1:10, 5:1 to 1:10, 4:1 to 1:10, 3:1 to 1:10, 2:1 to 1:10, 10:1 to 1:9, 10:1 to 1:8, 10:1 to 1:7, 10:1 to 1:6, 10:1 to 1:5, 10:1 to 1:4, 10:1 to 1:3, 10:1 to 1:2, or 1:1 to 3:1 in each fraction of the oligosaccharide preparation.
Item 107: Oligosaccharide preparation for use in treatment, amelioration and/or prophylaxis according to any one of items 59-106, wherein the ratio of 1,6-anhydro-β-D-glucofuranose to 1,6-anhydro-β-D-glucopyranose is about 10:1, 9:1, 8:1, 7:1, 6:1, 5:1, 4:1, 3:1, 2:1, 1:1, 1:2, 1:3, 1:4, 1:5, 1:6, 1:7, 1:8, 1:8, 1:9, or 1:10 in each fraction of the oligosaccharide preparation.
Item 108: Oligosaccharide preparation for use in treatment, amelioration and/or prophylaxis according to any one of items 59-107, wherein the ratio of 1,6-anhydro-β-D-glucofuranose to 1,6-anhydro-β-D-glucopyranose is about 2:1 in each fraction of the oligosaccharide preparation.
Item 109: Oligosaccharide preparation for use in treatment, amelioration and/or prophylaxis according to any one of items 59-108, wherein at least 1%, 5%, 10%, 20%, 30%, 40%, 50%, 60%, 70%, 80%, or 90% of anhydro-subunits in the oligosaccharide preparation are selected from a group consisting of 1,6-anhydro-β-D-glucofuranose and 1,6-anhydro-β-D-glucopyranose.
Item 110: Oligosaccharide preparation for use in treatment, amelioration and/or prophylaxis according to any one of items 59-109, wherein the weight average molecular weight of the oligosaccharide preparation is about from 300 to 5000 g/mol, 500 to 5000 g/mol, 700 to 5000 g/mol, 500 to 2000 g/mol, 700 to 2000 g/mol, 700 to 1500 g/mol, 300 to 1500 g/mol, 300 to 2000 g/mol, 400 to 1300 g/mol, 400 to 1200 g/mol, 400 to 1100 g/mol, 500 to 1300 g/mol, 500 to 1200 g/mol, 500 to 1100 g/mol, 600 to 1300 g/mol, 600 to 1200 g/mol, or 600 to 1100 g/mol.
Item 111: Oligosaccharide preparation for use in treatment, amelioration and/or prophylaxis according to any one of items 59-110, wherein the number average molecular weight of the oligosaccharide preparation is about from 300 to 5000 g/mol, 500 to 5000 g/mol, 700 to 5000 g/mol, 500 to 2000 g/mol, 700 to 2000 g/mol, 700 to 1500 g/mol, 300 to 1500 g/mol, 300 to 2000 g/mol, 400 to 1000 g/mol, 400 to 900 g/mol, 400 to 800 g/mol, 500 to 900 g/mol, or 500 to 800 g/mol.
Item 112: Oligosaccharide preparation for use in treatment, amelioration and/or prophylaxis according to any one of items 59-111, wherein the distribution of the degree of polymerization is determined and/or detected by MALDI-MS, GC-MS, LC-MS, SEC, HPLC and/or combination(s) thereof (e.g. MALDI-MS and SEC).
Item 113: Oligosaccharide preparation for use in treatment, amelioration and/or prophylaxis according to any one of items 59-112, wherein the degree of polymerization of the oligosaccharide preparation may be determined based on its molecular weight and molecular weight distribution.
Item 114: Use of an oligosaccharide preparation (e.g. a synthetic oligosaccharide preparation) to reduce the median relative abundance of Eimeria species in cecal digesta of a bird vaccinated against coccidiosis compared to a comparable control bird, wherein the comparable control bird was vaccinated against coccidiosis but not administered the oligosaccharide preparation, wherein the oligosaccharide preparation comprises at least n fractions of oligosaccharides each having a distinct degree of polymerization selected from 1 to n (DP1 to DPn fractions), wherein n is an integer greater than or equal to 2; and wherein each fraction comprises from at least about 0.5% to about 90% (e.g. from 1% to 90%; or e.g. from about 0.5% to about 15%) of anhydro-subunit containing oligosaccharides by relative abundance as determined by mass spectrometry.
Item 115: The use according to any one of items 1-3, 4-6, 7-58, wherein the median relative abundance of Eimeria species in cecal digesta of the bird vaccinated against coccidiosis is reduced compared to the comparable control bird (in particular, wherein the comparable control bird was vaccinated against coccidiosis but not administered the oligosaccharide preparation).
Item 116: The use according to item 114 or according to item 115, wherein the median relative abundance of Eimeria species in cecal digesta of the bird vaccinated against coccidiosis is reduced compared to the comparable control bird on at least one day within 42 days after vaccination against coccidiosis (e.g. on one or more day(s) selected from the group of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42 day(s) after vaccination against coccidiosis), preferably at least on day 24 after vaccination against coccidiosis.
Item 117: Oligosaccharide preparation (e.g. synthetic oligosaccharide preparation) for use in treatment, amelioration and/or prophylaxis against coccidiosis, wherein the oligosaccharide preparation is used in addition to a vaccination against coccidiosis, wherein the oligosaccharide preparation comprises at least n fractions of oligosaccharides each having a distinct degree of polymerization selected from 1 to n (DP1 to DPn fractions), wherein n is an integer greater or equal to 2; and wherein each fraction comprises from at least about 0.5% to about 90% (e.g. from 1% to 90%; or e.g. from about 0.5% to about 15%) of anhydro-subunit containing oligosaccharides by relative abundance as determined by mass spectrometry.
Item 118: The oligosaccharide preparation for use in treatment, amelioration and/or prophylaxis against coccidiosis according to item 117, wherein the oligosaccharide preparation is the oligosaccharide preparation according to any one of items 10-16, 18-58.
Item 119: The oligosaccharide preparation for use in treatment, amelioration and/or prophylaxis against coccidiosis according to any one of items 117 or 118, wherein the oligosaccharide preparation is comprised in a nutritional composition at an inclusion rate of at least 50 ppm (e.g. at least 50, 70, 100, 150, 200, 300, 400, 500 ppm), which nutritional composition is administered to a bird vaccinated against coccidiosis.
Item 120: The oligosaccharide preparation for use in treatment, amelioration and/or prophylaxis against coccidiosis according to any one of items 117-119, wherein the oligosaccharide preparation is administered to the bird before, after, and/or simultaneously with the diet of the bird vaccinated against coccidiosis, preferably simultaneously with the diet.

It is considered that the oligosaccharide preparation of the invention may be characterized by any combination of the individual features as described in the items above. For instance, a particular embodiment of the oligosaccharide preparation in a use of the invention may be characterized by a combination of the combined oligosaccharide preparation features as described in items 10, 15 and 25.

In the following, the present invention is further described by non-limiting examples. The present invention as disclosed herein is not limited to specific embodiments, figures, methodology, examples, protocols etc. described herein but solely defined by the claims.

### EXAMPLES

A feeding trial was performed to study the effects of oligosaccharide preparations on the performance of birds in husbandry. The test period began on Trial Day 0 (day of hatch of chicks), when chicks began being fed a commercial-type feed in pelleted form (further crumbled for Starter feeds), and ended on Trial Day 42. Each experimental unit contained 40 male broilers (Hubbard-Cobb) randomly assigned into 21 replicates per group for a total number of 840 animals per treatment on study. Broiler chicks were randomly assigned to treatments of Trial Day 0 (or on day of hatch) and were not replaced during the course of the trial. The chicks were observed daily for signs of unusual grow-out patterns or health problems. Body weights, feed consumption and feed conversion were measured on Trial Days 0, 10, 24, and 42. Cecal content samples, ileal tissue samples, and blood plasma samples were collected from 1 bird per pen at 24 and 42 days of age. For the vaccination program, all birds received Marek's vaccine, as well as being sprayed with vaccine against coccidiosis (COCCIVAC®-B52 by Merck Animal Health USA, which is a live oocysts vaccine isolated from chickens, prepared from anticoccidial-sensitive strains of E. acervulina, E. maxima, E. maxima MFP, E. mivati, and E. tenella according to the product bulletin) and for Newcastle bronchitis. No feed grade antibiotics were administered during the course of the study. All birds were grown on new litter. Feed and water were provided ad libitum throughout the conduct of the study.

The commercial-simulated test model employed in this study used broiler chicks (Gallus gallus domesticus) reared under a normal poultry industry Starter diet (0-10 days of age), Grower diet (11-24 days of age) and Finisher diet (25-42 days of age) at a floor space requirement of a minimum of 0.85 ft² per bird, reared in floor pens with new litter. Ration formulations were conducted via computer-generated linear regression program that simulates formulations conducted during practical poultry production techniques. Treatments were tested in male broilers. Broilers were continuously fed their experimental diets from time of placement on Trial Day 0 (day of hatch) to 42 days of age. All diets contained 1000 FYT/kg of phytase (RONOZYME® HiPhos).

| | | Test Material |
|---|---|---|
| | | Inclusion |
| Treatment | Description | (ppm) |
| Control | high/poor quality protein | 0 |
| Test | Control + oligosaccharide | 500 |
| | preparation | |

Broiler chicks were weighed and randomly placed into each pen on day of hatch (Trial Day 0) and fed their respective diets. Each pen had sufficient floor density, feeder and waterer space for each grow-out area for chickens up to 42 days of age. Following 42 days of grow-out, broilers were weighed, feed consumption determined, and feed conversion ratio (feed consumed/body weight) calculated and adjusted for mortality.

Oligosaccharide preparations comprise at least n fractions of oligosaccharides each having a distinct degree of polymerization selected from 1 to n (DP1 to DPn fractions), wherein n is an integer greater than 3; wherein each of a DP1 and DP2 fraction independently comprises from about 0.5% to about 15% of anhydro-subunit containing oligosaccharides by relative abundance as determined by mass spectrometry. Oligosaccharide preparations were produced as disclosed in WO 2020/097458, WO 2016/007778.

Test material description: Test material was provided in either liquid or powder form, and mixed into the treatment feeds. The treatment feeds were then pelleted (and further crumbled for the Starter feeds) and placed into the pens according to the pen design for this study. Treatments were fed continuously from Trial Days 0-42. Test material treatments (comprising oligosaccharide preparation according to the invention) were compared to a Control treatment (not comprising oligosaccharide preparation according to the invention).

Experimental design: A total of 8,000 male broiler chicks (a sufficient number to ensure availability of at least 7,560 healthy male chicks for the conduct of the study) were obtained from a commercial hatchery on Trial Day 0 (same as hatch date). These were immediately transported to the feeding trial facility under temperature-controlled conditions to assure bird comfort. After arrival at the facility, broilers were immediately randomized. There were 40 healthy/viable male broilers per pen with 21 pens per test group for a total of 840 broilers per treatment group. Broilers were fed their respective treatment feed ad libitum from day of hatch (Trial Day 0) to 42 days of age.

Detailed broiler chick description: Animal care practices conformed to the Guide for the Care and Use of Agricultural Animals in Agricultural Research and Teaching (FASS, 2010, 3rd Edition). Commercial broilers (Hubbard-Cobb) were obtained at hatch (Trial Day 0) from a commercial hatchery. Broilers were evaluated upon receipt for signs of disease or other complications that may have affected the outcome of the study. Following examination, broilers were weighed. Broilers were allocated to each pen and to treatment groups using a randomized block design. Weight distribution across the treatment groups were assessed prior to feeding by comparing the individual test group standard deviations of the mean against that of the control group. Differences between control and test groups were within one standard deviation, and as such, weight distribution across treatment groups were considered acceptable for this study.

Broiler chicks (on day of hatch, called Day 0) were collected in the early morning and were randomly assigned to each experimental pen within 12 hours of hatch. Weak birds were removed and humanely sacrificed. Birds were not replaced during the study.

Housing and daily observations: Each experimental test unit of broiler mixed-sex chicken pens were housed in separated pens, located in a room containing forced air heaters with a cross-house ventilation system. Broilers were placed in a 5 ft×10 ft pen floor area with a minimum of 0.85 ft² per bird (without feeder and waterer space) provided. At least two nipple drinkers per pen (via well water) were provided.

Feeders were employed for the grow-out period and checked daily to ensure that all birds had access to feed at all times.

The light program employed made use of incandescent lighting for approximately 23 hours of continuous light and 1 hour of darkness per day for Days 0-7, and for approximately 20 hours of continuous light and 4 hours of darkness per day for the remainder of the study.

Birds were observed daily for overall health, behavior and/or evidence of toxicity, and environmental conditions. Temperature in the test facility was checked daily. Drinking water and feed were confirmed to be provided ad libitum.

No type of medication (other than test material) was administered during the entire feeding period. Mortalities were collected daily and body weights recorded on all broilers found dead or moribund.

Data and observations: Live performance body weights and feed intakes were collected on Days 0, 10, 24, and 42 during the growing period. Weight gain, feed intake, feed:gain ratio (feed efficiency) were calculated for 0-42 days of age and other age periods between hatch and market weights. Differences between broilers fed control and test groups were statistically evaluated at P<0.05 in a typical ANOVA analysis of variance test model, employing Treatment x Replicate RCB (Randomized Complete Block). Control group was considered to be the following: Treatment 1, with no added test materials.

At the end of the study, all carcasses of necropsied broilers and all birds remaining at the end of the study, after being humanely euthanized, were disposed of according to local regulations via on-farm composting techniques.

Diet Preparation: A basal ration for each phase was formulated to meet or exceed minimum nutrient requirements of a typical commercial broiler diet using formulations employed by qualified nutritionist with training in poultry feed formulations, and formulated rations met or exceeded NRC Nutrient Requirements for Poultry as a guideline (9th edition, 1994). Feed formulations were furnished by a veterinarian, conducted by a regression analysis program commonly used for Least-Cost Feed Formulation in the poultry industry. Test materials were then mixed into the basal ration.

Dietary protein, lysine, methionine, methionine+cystine, arginine, threonine, tryptophan, total phosphorus, available phosphorus, total calcium, dietary sodium, and dietary choline were met by adjusting the concentrations of corn and soybean meal ingredients, as well as other minor ingredients commonly used in poultry production. Mixing equipment was flushed with ground corn prior to each diet preparation. All diets were prepared using a paddle mixer. The mixer was cleaned between each diet using compressed air and vacuum, mixing equipment was flushed with ground corn between each treatment group, and flush material was retained for disposal.

Diet and water administration: Diets were fed in three feed phases: Starter diet (0-10 days of age), Grower diet (11-24 days of age) and Finisher diet (25-42 days of age). All diets were offered ad libitum, without restriction. Fresh well water (from the research facility deep well) was provided ad libitum.

Feed Formulation Parameters:

| | Starter | Grower | Finisher |
|---|---|---|---|
| Ingredients, % | (d 0-11) | (d 11-24) | (d 24-42) |
| Corn | 58.353 | 65.383 | 69.413 |
| SBM, 47.9% CP | 29.992 | 20.404 | 16.879 |
| Anim & veg fat | 0.05 | 0.05 | 0.050 |
| DL-methionine | 0.515 | 0.4.77 | 0.420 |
| Salt | 0.342 | 0.332 | 0.332 |
| L-Lysine-HCL | 0.367 | 0.439 | 0.400 |
| Limestone | 1.794 | 1.537 | 1.470 |
| Dical Phos | 1.079 | 0.869 | 0.739 |
| Choline CL - 70% | 0.044 | 0.080 | 0.091 |
| TM & Vit Premix | 0.075 | 0.075 | 0.075 |
| Wheat midds | 2.377 | 0.344 | 0.121 |
| Corn DDGs | 5.0 | 10.00 | 10.00 |
| Ronozyme HiPhos | 0.01 | 0.01 | 0.01 |
| Nutrient | Amount | Amount | Amount |
| Protein, % | 20.3 | 17.5 | 16.0 |
| Ca, % | 0.920 | 0.770 | 0.720 |
| Tphos, % | 0.656 | 0.578 | 0.533 |
| Avphos, % | 0.350 | 0.300 | 0.270 |
| Sodium, % | 0.160 | 0.160 | 0.160 |
| ME kcal/kg | 2900 | 3040 | 3084 |
| Av Lys, % | 1.33 | 1.140 | 1.010 |
| Av | 0.99 | 0.880 | 0.790 |
| M + C, % | | | |
| Choline, g/kg | 1.60 | 1.60 | 1.60 |

Measurement and sampling schedule: On days 0, 10, 24 and 42: Performance; BWG, FI and FCR (corrected and uncorrected for mortality) Per pen basis. On days 24 and 42: Cecal samples (1 bird/pen), 21 reps/trt; Ileal tissue (1 bird/pen), 21 reps/trt; Plasma (1 bird/pen), 21 reps/tht. On day 0 (before bird placement) and day 42: Utter samples (one composite sample per pen), 21 reps/trt (3 in front, 3 in the middle, and 3 in the back).

Results: The test period began on Trial Day 0 (day of hatch of chicks), and chicks were fed a commercial-type feed in pelleted form (crumbles on Days 0-10) until the end of the study. Each treatment contained 21 replicates per treatment randomly assigned and containing 40 male broilers per replicate. Chicks were randomly assigned to treatments on Trial Day 0 (or day of hatch). At 42 days of age, live performance (growth weight gain, mortality and feed conversion) and other criteria were determined.

With respect to daily observations, each pen was closely monitored each day to determine overall health, bird behavior and/or evidence of toxicity, and environmental conditions. Temperature was checked within the growing area employed for this study daily. Temperature program employed for this study was maintaining temperatures of approximately 86+/−5° F. for the first seven (7) days, decreasing approximately 1° F. per day thereafter until a target of approximately 70+/−5° F. was reached, which was maintained throughout the study.

For the entire grow-out period (Days 0-42), body weight gain showed significant improvement over the Control group when broilers were fed diets containing the oligosaccharide preparation Feed conversion for Trial Days 0-42 followed a similar patter as final body weights. Mortality was considered average for this breed in all groups throughout the growing period to 42 days of age without significant differences. Normal poultry industry mortality is typically <4.5% when birds are grown on litter bedding floors.

Observed data on average body weight, feed conversion ratio (corrected for mortality), mortality in %, and average body weight gain is shown in the subsequent table. Statistical evaluation is for each observation is shown in the respective row below.

| Data | Control | Test |
|---|---|---|
| Average body weight in kg, Day 10 | 0.3623 | 0.3684 |
| Statistical evaluation¹ | ^{a} | ^{a} |
| Feed conversion ratio (corrected), Day 0-10 | 1.167 | 1.152 |
| Statistical evaluation¹ | ^{a} | ^{b} |
| Mortality in %, Day 0-10 | 1.19 | 1.31 |
| Statistical evaluation¹ | ^{a} | ^{a} |
| Average body weight gain in kg, Day 0-10 | 0.3184 | 0.3248 |
| Statistical evaluation¹ | ^{a} | ^{a} |
| Average body weight in kg, Day 24 | 1.0484 | 1.0756 |
| Statistical evaluation¹ | ^{a} | ^{b} |
| Feed conversion ratio (corrected), Day 0-24 | 1.413 | 1.379 |
| Statistical evaluation¹ | ^{a} | ^{b} |
| Mortality in %, Day 0-24 | 2.262 | 2.857 |
| Statistical evaluation¹ | ^{a} | ^{a} |
| Average body weight gain in kg, Day 0-24 | 1.0045 | 1.0319 |
| Statistical evaluation¹ | ^{a} | ^{b} |
| Average body weight in kg, Day 42 | 2.5071 | 2.5755 |
| Statistical evaluation¹ | ^{a} | ^{b} |
| Feed conversion ratio (corrected), Day 0-42 | 1.833 | 1.783 |
| Statistical evaluation¹ | ^{a} | ^{b} |
| Mortality in %, Day 0-42 | 3.297 | 4.762 |
| Statistical evaluation¹ | ^{a} | ^{a} |
| Average body weight gain in kg, Day 0-42 | 2.4632 | 2.5318 |
| Statistical evaluation¹ | ^{a} | ^{b} |
| Feed conversion ratio (corrected), Day 10-24 | 1.529 | 1.486 |
| Statistical evaluation¹ | ^{a} | ^{b} |
| Mortality in %, Day 10-24 | 1.09 | 1.579 |
| Statistical evaluation¹ | ^{a} | ^{a} |
| Average body weight gain in kg, Day 10-24 | 0.6861 | 0.7071 |
| Statistical evaluation¹ | ^{a} | ^{b} |
| Feed conversion ratio (corrected), Day 24-42 | 2.136 | 2.075 |
| Statistical evaluation¹ | ^{a} | ^{b} |
| Mortality in %, Day 24-42 | 1.003 | 1.886 |
| Statistical evaluation¹ | ^{a} | ^{a} |
| Average body weight gain in kg, Day 24-42 | 1.4587 | 1.4999 |
| Statistical evaluation¹ | ^{a} | ^{b} |

¹Means within a row without a common superscript are significantly different (P < 0.05) as determined by Least Significant Difference.

Microbiome analysis: Cecal digesta samples were collected from the control group as well as from the test group (1 bird/pen and 21 replicates/treatment). Cecal samples were kept frozen at −80° C. before DNA extraction for metagenomics analysis. Metagenomic DNA was extracted using DNeasy PowerSoil HTP96 following manufacturer instructions (Qiagen, Germany). A person having skill in the art is aware of suitable methods for extracting metagenomic DNA. DNA was sequenced on an Illumina HiSeq 3000 apparatus with a target depth of 5 GB per sample. In order to choose appropriate filtering and trimming parameters, the raw fastq files from shallow shotgun sequencing were inspected using FastQC v0.11.5 (Andrews, S, FastQC: a quality control tool for high throughput sequence data. 2010, Babraham Bioinformatics, Babraham Institute, Cambridge, United Kingdom). Based on the quality reports, Cutadapt (Martin, M., Cutadapt removes adapter sequences from high-throughput sequencing reads. EMBnet.journal; Vol 17, No 1: Next Generation Sequencing Data AnalysisDO −431 10.14806/ej.17.1.200, 2011) was used to trim the first 10 bases of each read, shorten each read to a maximum of 130 bp, and discard any read less than 120 bp long. This removed any remaining adapter fragments and eliminated regions near the end of the read where the quality dropped, confirmed by another quality report from FastQC. Taxonomic profiles of the demultiplexed reads and taxa relative abundance estimates were generated using Kraken2 (Wood et al. 2019. Genome Biology 20:257) and Bracken (Lu et al. 2017. PeerJ Computer Science 3:e103) bioinformatic tools. On day 24 after hatch, the relative abundance of microorganisms from the genus Eimeria was found decreased in the test group compared to the control group (P=0.088). Specifically, on day 24 after hatch the median relative abundance of Eimeria species was found decreased by 41.31% in the test group compared to the control group. On day 42 after hatch the median relative abundance of Eimeria species was found decreased by 23.17% in the test group compared to the control group. It was thus concluded that the use of the oligosaccharide preparation described herein not only improved the performance (increased average body weight and/or decreased FCR, increased average body weight gain) of animals vaccinated against coccidiosis, but also supported the anti-coccidiosis effect of the vaccination itself.

1. Use of an oligosaccharide preparation, e.g. a synthetic oligosaccharide preparation, to reduce the relative abundance of microorganisms from the genus Eimeria in a bird,
wherein the oligosaccharide preparation comprises at least n fractions of oligosaccharides each having a distinct degree of polymerization selected from 1 to n (DP1 to DPn fractions), wherein n is an integer greater than or equal to 2; and
wherein each fraction comprises from at least about 0.5% to about 90% (e.g. from 1% to 90%; or e.g. from about 0.5% to about 15%) of anhydro-subunit containing oligosaccharides by relative abundance as determined by mass spectrometry.

2. Use of an oligosaccharide preparation, e.g. a synthetic oligosaccharide preparation, to support, in particular to increase, the anti-coccidiosis effect of a vaccination against coccidiosis,
wherein the oligosaccharide preparation comprises at least n fractions of oligosaccharides each having a distinct degree of polymerization selected from 1 to n (DP1 to DPn fractions), wherein n is an integer greater than or equal to 2; and
wherein each fraction comprises from at least about 0.5% to about 90% (e.g. from 1% to 90%; or e.g. from about 0.5% to about 15%) of anhydro-subunit containing oligosaccharides by relative abundance as determined by mass spectrometry.

3. Use of an oligosaccharide preparation, e.g. a synthetic oligosaccharide preparation, to increase average body weight and/or to decrease feed conversion ratio and/or to increase average body weight gain of a bird vaccinated against coccidiosis,
wherein the oligosaccharide preparation comprises at least n fractions of oligosaccharides each having a distinct degree of polymerization selected from 1 to n (DP1 to DPn fractions), wherein n is an integer greater than or equal to 2;
wherein each fraction comprises from at least about 0.5% to about 90% (e.g. from 1% to 90%; or e.g. from about 0.5% to about 15%) of anhydro-subunit containing oligosaccharides by relative abundance as determined by mass spectrometry; and
wherein the average body weight of the bird is increased and/or the feed conversion ratio of the bird is decreased and/or the average body weight gain of the bird is increased, all in comparison to a comparable control bird, wherein the comparable control bird was not administered the oligosaccharide preparation.

4. The use according to claim 3, wherein the average body weight of the bird is increased and/or the feed conversion ratio of the bird is decreased and/or the average body weight gain of the bird is increased on at least one day within 42 days after vaccination against coccidiosis (e.g. on one or more day(s) selected from the group of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42 day(s) after vaccination against coccidiosis).

5. The use according to claim 4, wherein the average body weight is increased by at least 0.2% (e.g. at least 0.5%, 0.75%, 1.00%, 1.25%, 1.50%, 1.68%, 1.70%, 2.0%, 2.5%, 2.7% etc.); and/or wherein the feed conversion ratio is decreased by at least 0.2% (e.g. at least 0.5%, 0.75%, 1.00%, 1.25%, 1.50%, 1.7%, 2.0%, 2.4%, 2.5%, 2.7%, 2.8% etc.); and/or wherein the average body weight gain is increased by at least 0.2% (e.g. at least 0.5%, 0.75%, 1.0%, 1.5%, 2%, 2.5%, 2.7%, 2.8%, 3.0%)
on at least one day within 42 days after vaccination against coccidiosis (e.g. on one or more day(s) selected from the group of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42 day(s) after vaccination against coccidiosis).

6. Use of an oligosaccharide preparation, e.g. synthetic oligosaccharide preparation, to adapt the average body weight and/or the feed conversion ratio and/or the average body weight gain of a bird vaccinated against coccidiosis to match the average body weight and/or the feed conversion ratio and/or the average body weight gain of a bird not vaccinated against coccidiosis;
wherein the oligosaccharide preparation comprises at least n fractions of oligosaccharides each having a distinct degree of polymerization selected from 1 to n (DP1 to DPn fractions), wherein n is an integer greater than or equal to 2; and
wherein each fraction comprises from at least about 0.5% to about 90% (e.g. from 1% to 90%; or e.g. from about 0.5% to about 15%) of anhydro-subunit containing oligosaccharides by relative abundance as determined by mass spectrometry.

7. The use according to claim 6, wherein the average body weight and/or the feed conversion ratio and/or the average body weight gain of the bird vaccinated against coccidiosis matches the average body weight and/or the feed conversion ratio and/or the average body weight gain of a bird vaccinated against coccidiosis of the bird not vaccinated against coccidiosis on at least one day within 42 days after vaccination against coccidiosis (e.g. on one or more day(s) selected from the group of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42 day(s) after vaccination against coccidiosis).

8. The use according to claim 6, wherein the average body weight of the bird not vaccinated against coccidiosis is at least 0.2% (e.g. at least 0.5%, 0.75%, 1.00%, 1.25%, 1.50%, 1.68%, 1.70%, 2.0%, 2.5%, 2.7% etc.) higher than the average body weight of a bird vaccinated against coccidiosis and not administered the oligosaccharide preparation; and or the feed conversion ratio of the bird not vaccinated against coccidiosis is at least 0.2% (e.g. at least 0.5%, 0.75%, 1.00%, 1.25%, 1.50%, 1.7%, 2.0%, 2.4%, 2.5%, 2.7%, 2.8% etc.) lower than the feed conversion ratio of a bird vaccinated against coccidiosis and not administered the oligosaccharide preparation; and/or the average body weight gain of a bird not vaccinated against coccidiosis is at least 0.2% (e.g. at least 0.5%, 0.75%, 1.0%, 1.5%, 2%, 2.5%, 2.7%, 2.8%, 3.0%) higher than the average body weight gain of a bird vaccinated against coccidiosis and not administered the oligosaccharide preparation
on at least one day within 42 days after vaccination against coccidiosis (e.g. on one or more day(s) selected from the group of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42 day(s) after vaccination against coccidiosis).

9. The use according to claim 1, wherein the oligosaccharide preparation is comprised in a nutritional composition administered to the bird.

10. The use according to claim 9, wherein the oligosaccharide preparation is comprised in the nutritional composition at an inclusion rate of at least 50 ppm (e.g. at least 50, 70, 100, 150, 200, 300, 400, 500 ppm).

11. The use according to claim 1, wherein the bird is poultry (e.g. chicken, duck, goose, turkey, guinea fowl, pigeon, quail), preferably chicken.

12. The use according to claim 1, wherein n is at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100.

13. The use according to claim 1, wherein at least one fraction of the oligosaccharide preparation comprises less than 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, or 2% anhydro-subunit containing oligosaccharides by relative abundance; and/or wherein each fraction of the oligosaccharide preparation comprises greater than 0.2%, 0.5%, 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, or 80% anhydro-subunit containing oligosaccharides by relative abundance.

14. The use according to claim 1, wherein the oligosaccharide preparation has a weight average molecular weight from about 300 to 5000 g/mol (e.g. from about 2000 to 2800 g/mol, 2100 to 2700 g/mol, 2200 to 2600 g/mol, 2300 to 2500 g/mol, or 2320 to 2420 g/mol), 500 to 5000 g/mol, 700 to 5000 g/mol, 500 to 2000 g/mol, 700 to 2000 g/mol, 700 to 1500 g/mol, 300 to 1500 g/mol, 300 to 2000 g/mol, 400 to 1300 g/mol, 400 to 1200 g/mol, 400 to 1100 g/mol, 500 to 1300 g/mol, 500 to 1200 g/mol, 500 to 1100 g/mol, 600 to 1300 g/mol, 600 to 1200 g/mol, or 600 to 1100 g/mol; and/or wherein the oligosaccharide preparation has a number average molecular weight from about 1000 to 2000 g/mol, 1100 to 1900 g/mol, 1200 to 1800 g/mol, 1300 to 1700 g/mol, 1400 to 1600 g/mol, or 1450 to 1550 g/mol.

15. The use according to claim 1, wherein the relative abundance of oligosaccharides in each of the n fractions of the oligosaccharide preparation decreases monotonically with its degree of polymerization.

16. Oligosaccharide preparation, e.g. synthetic oligosaccharide preparation, for use in treatment, amelioration and/or prophylaxis of a bird vaccinated against coccidiosis,
wherein the oligosaccharide preparation comprises at least n fractions of oligosaccharides each having a distinct degree of polymerization selected from 1 to n (DP1 to DPn fractions), wherein n is an integer greater or equal to 2; and
wherein each fraction comprises from at least about 0.5% to about 90% (e.g. from 1% to 90%;
or e.g. from about 0.5% to about 15%) of anhydro-subunit containing oligosaccharides by relative abundance as determined by mass spectrometry.

17. Oligosaccharide preparation, e.g. synthetic oligosaccharide preparation, for use in treatment, amelioration and/or prophylaxis of at least one adverse effect of vaccination against coccidiosis in a bird vaccinated against coccidiosis,
wherein the oligosaccharide preparation comprises at least n fractions of oligosaccharides each having a distinct degree of polymerization selected from 1 to n (DP1 to DPn fractions), wherein n is an integer greater or equal to 2; and
wherein each fraction comprises from at least about 0.5% to about 90% (e.g. from 1% to 90%;
or e.g. from about 0.5% to about 15%) of anhydro-subunit containing oligosaccharides by relative abundance as determined by mass spectrometry.

18. Oligosaccharide preparation, e.g. synthetic oligosaccharide preparation, for use in treatment, amelioration and/or prophylaxis against coccidiosis,
wherein the oligosaccharide preparation is used in addition to a vaccination against coccidiosis,
wherein the oligosaccharide preparation comprises at least n fractions of oligosaccharides each having a distinct degree of polymerization selected from 1 to n (DP1 to DPn fractions), wherein n is an integer greater or equal to 2; and
wherein each fraction comprises from at least about 0.5% to about 90% (e.g. from 1% to 90%;
or e.g. from about 0.5% to about 15%) of anhydro-subunit containing oligosaccharides by relative abundance as determined by mass spectrometry.
